(19) United States
(12) Patent Application Publication
(10) Pub. No.: US 2026/0241064 A1
(43) Pub. Date: Aug. 20, 2026

(54) NATURAL FLUORESCENT SILK FIBROIN FOR BIOMEDICAL APPLICATIONS

(71) Applicant: David Wu, San Jose, CA (US)

(72) Inventor: David Wu, San Jose, CA (US)

(21) Appl. No.: 19/216,629

(22) Filed: May 22, 2025

Related U.S. Application Data
(60) Provisional application No. 63/760,571, filed on Feb. 19, 2025.

(51) Int. Cl. A61L 26/00; A01K 67/35; A61L 27/16; A61L 27/22; A61L 27/52

(52) U.S. Cl. CPC A61L 26/0028; A01K 67/35; A61L 26/0014; A61L 26/008; A61L 27/16; A61L 27/227; A61L 27/52; A61L 2300/442

(57) ABSTRACT

The present invention relates to synthesize nontoxic fluorescent silk fibroin for biomedical applications, including wound healing and cell growth support. Fluorescent silk fibroin is produced by incorporating nontoxic carbon quantum dots (CQDs) into the diet of Bombyx mori silkworms. CQDs are synthesized from Rhodamine B using a hydrothermal method, with particle sizes ranging from 2-3 nm. When fed to silkworms at a concentration of 0.1 wt %, CQDs enhance the mechanical and optical properties of the resulting fluorescent silk. The fluorescent silk fibroin is further processed into scaffolds and hydrogel films for biomedical applications. Experimental results demonstrate increased mesenchymal stem cell adhesion and proliferation, with cell coverage increasing from 9% to 37.5% over three days. Additionally, fluorescent silk fibroin bandages enable real-time monitoring of wound healing, as fluorescence intensity decreases with healing progression. This invention provides a nontoxic alternative for biomedical applications in tissue engineering and regenerative medicine.

### FIELD OF THE INVENTION

This invention pertains to the preparation of fluorescent silk fibroin by incorporating nontoxic carbon quantum dots (CQDs) into the diet of Bombyx mori silkworms. It further explores the biomedical applications of fluorescent silk fibroin in wound healing and scaffold systems that support cell growth and adhesion.

### BACKGROUND OF THE INVENTION

Fluorescent silk can be obtained through three main methods: artificial modification, genetic modification, and feeding silkworms with fluorescent compounds. Artificial chemical modification requires various chemical reagents to change the fluorescence of the silk after it has already been produced. This can have a complex process, harm the silk's structure and properties, and have suboptimal modification effects. Genetic modification is done by modifying the genes of silkworms so that they can show fluorescence. While effective, the downside of this method is that it can ruin the gene pool of the silkworm and requires a lot of human labor. The third way is naturally feeding fluorescent materials to silkworms, allowing them spin fluorescent cocoons. This method is inexpensive, requires less human labor, and allows silkworms to spin colorful cocoons without harming the structure.

Currently, two main types of fluorescent materials are used to produce fluorescent silk by feeding them to silkworms: Rhodamine derivatives (Rhodamine B, 101, 110 etc.) and nanomaterials (Graphene QDs, CdSe/ZnS QDs, CdTe QDs etc.). Rhodamine derivatives are widely used due to their low cost, high quantum yield, and strong fluorescence across various wavelengths. However, their toxicity limits their applications in the biomedical field. Nanomaterials like carbon nanodots and quantum dots have been fed to silkworms and were shown to enhance silk fibroin's strength, expanding biomedical applications. Cheng et al. (“Quantum dots-reinforced luminescent silkworm silk with superior mechanical properties and highly stable fluorescence.” Journal of Materials Science, vol. 54, no. 13, July 2019, pp. 9945) found that quantum dot-treated silk exhibited 1.5 to 2 times higher tensile strength than control silk, whereas Rhodamine B-treated silk showed lower strength. While the nanomaterials can enhance silk's properties, they have a very low absorption rate within silkworms. This results in fluorescent silk with super weak fluorescence, which also limits its abilities in the biomedical field.

To address the shortage of suitable fluorescent materials to produce nontoxic and high fluorescent intensity silk for biomedical applications, this research aims to (1) synthesize nontoxic carbon quantum dots with high fluorescent intensity; (2) develop a method to feed the CQDs to silkworms to naturally produce fluorescent silk with strong fluorescence and high mechanical strength; and (3) apply the silk in biomedical applications, including its use as cell growth scaffolds and silk fibroin bandage for wound healing in a mouse model.

### SUMMARY OF THE INVENTION

Fluorescent silk fibroin, which is produced by modifying the diet of the Bombyx mori, has recently emerged as a promising material for biomedical applications. However, there is a lack of research on nontoxic fluorescent materials that meet high levels of biomedical safety. In this study, three types of nontoxic carbon quantum dots (CQD) were synthesized from Rhodamine B using the hydrothermal synthesis method. The size of the CQDs ranged from 2-3 nm, and all had shown bright fluorescence. The three carbon quantum dots (O-CQD, G-CQD, B-CQD) were fed to silkworms at a concentration of 0.1 wt % in silkworm chow to produce nontoxic fluorescent silk with enhanced mechanical and optical properties. The fluorescent silk was fabricated into silk fibroin scaffolds and films and allowed for two biomedical applications. First, there is notable mesenchymal stem cell adhesion and growth on cell culture scaffolds, with cell area increasing from 9%-37.5% over three days. Secondly, fluorescent bandages have been designed for wound healing, with fluorescence intensity reducing on healing. This study makes the following contributions. It successfully synthesizes three nontoxic carbon quantum dots (CQDs) using a hydrothermal method. Using the advanced materials, this study successfully produces fluorescent cocoons by incorporating CQDs into the silkworms'diet. Furthermore, this study demonstrates two key applications-the development of a CQD-loaded silk fibroin scaffold that supports cell growth and adhesion, and the fabrication of a fluorescent silk fibroin bandage that enhances wound healing.

### DETAILED DESCRIPTION OF THE INVENTION

### Synthesis of Carbon Quantum Dots

Carbon quantum dots were derived from Rhodamine B through a hydrothermal method. Blue CQDs were derived by putting Rhodamine B (1.5 mg) and urea (5 g) in a 100 mL beaker with 10 mL of distilled water. After sonication, the mixture was transferred into a 100 mL autoclave linear to perform hydrothermal treatment at 180° C. for 10 hours. Green CQDs were synthesized by mixing Rhodamine B (0.25 g), sodium hydroxide (0.4 g), and 10 ml of water in a 100 mL beaker. After sonication, it was transferred to a 100 mL autoclave linear to conduct hydrothermal treatment at 180° C. for 10 hours. Orange CQDs were synthesized by mixing Rhodamine B (0.25 g) and 10 ml of water into a 100 mL beaker. This was then transferred to an autoclave linear and hydrothermal treatment was conducted at 240° C. for 13 hours. The resulting Orange, Green, and Blue CQDs all showed bright fluorescence with emission peaks at 600 nm, 550 nm, and 471 nm respectively. They had excitation peaks at 400 nm, 370 nm, and 370 nm respectively.

### Culture of Silkworms and Degumming Process

The normal feed was prepared by mixing 20 grams of mulberry powder with 60 mL of hot water. The mixture was thoroughly stirred, microwaved at 1000 watts for 25 seconds, and then stirred again for an additional 15 seconds. The feed was then left to cool down before being fed to the silkworms. The modified feed was prepared by incorporating 0.1 wt % of fluorescent material with 20 grams of mulberry powder. Subsequently, 60 mL of water was added, and the mixture was microwaved at 1000 watts for 25 seconds, and then stirred again for an additional 15 seconds. Silkworm eggs were purchased from Mulberry Farms. The silkworms were raised at room temperature (23-28° C.) and fed a standard diet of silkworm chow. They were divided into 10 groups of 50 silkworms each, with each group assigned a distinct type of feed for testing. Initially, all silkworms were fed a normal diet until the third day of the fifth instar, after which each group received a modified diet until they spun their cocoons. The silk cocoons were first degummed using 0.04M NaHCO₃ with 1.5 mL of alcalase for 6 hours at 40° C. The silk was then left to dry overnight.

### Analysis of Fluorescent Silk

The Carbon Quantum Dots that were synthesized were first mixed into an aqueous solution. When the solutions were placed under 365 nm UV light, the solutions exhibited bright fluorescence.

Photoluminescence Spectroscopy spectrashows that all three CQDs had emission peaks, proving that they contained fluorescence. The Orange CQDs were excited under 400 nm and showed an emission peak of 600 nm, Green CQDs were excited under 370 nm light and showed an emission peak of 550 nm, and Blue CQDs were excited under 370 nm light and showed an emission peak of 471 nm. It was found that the Orange Carbon Quantum Dots showed a significantly higher intensity of fluorescence compared to the Green CQDs and Blue CQDs. This was most likely due to the different concentrations of Rhodamine in the solution during the preparation phase, as Orange CQDs only required distilled water to produce.

Transmission Electron Microscopy (TEM) images were captured to confirm the successful synthesis of the CQDs. The CQDs were placed on a copper grid and loaded into the TEM for imaging. ImageJ analysis of the images revealed that Blue CQDs had the smallest average diameter of about 2.64 nm, Green CQDs measured about 2.73 nm, and Orange CQDs had the largest diameter at about 2.91 nm.

The quantum yields (QY) of the CQDs were determined by dissolving them in deionized water and measuring their absolute QY using an integrating sphere. The results revealed that Blue CQDs had a QY of 4.18%, Green CQDs exhibited a significantly higher QY of 76.4%, and Orange CQDs had a QY of 59.2%. These findings highlight the strong fluorescence efficiency of the Green and Orange CQDs.

### Property Measurement of Fluorescent Silk

To study the finer structures of the silk produced, various tests were conducted.

SEM images of fluorescent silk were captured. The samples were coated with 10 nm of platinum and images were taken at 800× magnification. From the SEM images, it was observed that the silk produced by the Carbon Quantum Dots produced a more consistent silk with a thicker structure. In contrast, the silk produced by Rhodamine showed a slightly thinner structure and had more inconsistencies. The silk's structure consists of two main components: sericin and fibroin. Sericin is a substance that surrounds the fibroin and is removed during the degumming process. The cross-sectional SEM image of the silkhighlights both the silk fibroin and sericin components. The left side of the silk was not degummed while the right side went through the degumming process. Within the fibroin, there consists of microfibrils and nanofibrils. These are the structural elements that make up the silks fibers and allow it to have strong mechanical properties.

To study the structure of the different fluorescent silk produced by different fluorescent materials, size distribution tests were conducted from SEM images using ImageJ. From the size distribution test, it was found that silk produced by Carbon Quantum Dots had a slightly thicker structure compared to silk produced by Control group and Rhodamine materials. Carbon Quantum Dots showed an average diameter of around 14.25 nm, Control group showed an average diameter of around 12.02 nm, while Rhodamine materials showed a diameter of around 10.77 nm. The CQDs showed a slightly thicker silk structure.

To test the strength of the fluorescent silk produced, mechanical strength tests were conducted using a strength testing machine. The silk was wrapped around the hook of the machine and slowly stretched until it broke. From the strength test experiment, it was found that the silk produced by the Carbon Quantum Dots (CQDs) exhibited a strength of 1.16 N, while Rhodamine-derived silk had a strength of 0.56 N, and the control group had a strength of 0.89 N. The silk produced by CQDs showed an approximate 130% increase in strength compared to the control group, while the silk produced by Rhodamine only showed about 62.9% of the control group's strength. The stronger mechanical strength of the CQD silk is most likely attributed to the increased silk thickness observed in the previous experiment, as the larger diameter contributes to enhanced strength.

### Fluorescent Silk Fibroin Scaffold

To test the applications of the nontoxic fluorescent silk fibroin in the biomedical field, two key biomedical applications were demonstrated. First, a silk fibroin scaffold was developed by wrapping degummed silk across a glass coverslip. From the cell culture experiment, it was found that the cells were able to grow and proliferate within the scaffold. By the end of the 6^{th} day period, cells covered the scaffold and successfully grew on top of the silk. The 3D fluorescent confocal imaging revealed that the cells were distinguishable from the fluorescent silk, highlighting its potential as a promising material for bioimaging applications. ImageJ was used to calculate the area percentage of cells on the scaffold. The total area occupied by the cells was outlined and divided by the total area to obtain a percentage. It was found that the cell area increased from 9% to 37.5% over the course of three days. The results indicate that fluorescent silk scaffolds offer several benefits in tissue engineering and regenerative medicine. First, the scaffold provides a 3D framework that closely resembles the natural extracellular matrix (ECM), facilitating cell growth and tissue development. This enables the scaffold to exhibit properties such as cell proliferation and adhesion. Another benefit of the fluorescent scaffold is that its fluorescent properties allow for real-time visualization of cellular interactions with the scaffold, tracking of cell growth, and monitoring of scaffold degradation over time.

### Fluorescent Silk Fibroin Bandage

The second key biomedical application demonstrated was a wound healing test conducted on the back of a mouse. To conduct this, a nontoxic fluorescent silk fibroin bandage was developed from silk fibroin.

To test the feasibility of implementing silk fibroin as a material for wound healing, hydrogel films were made from fluorescent silk and a biocompatible polymer selected from polyvinyl alcohol, polyethylene oxide (PEO), polyethylene glycol (PEG), fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans and polyanhydrides and mixture thereof. The amount of fluorescent silk is from 0.01 to 30% by weight of hydrogel film, preferably from 0.1 to 10% by weight of hydrogel film; the amount of the biocompatible polymer is from 0.1 to 20% by weight of hydrogel film, preferably from 1-10% by weight of hydrogel film. First, the silk solution was prepared by dissolving 10 grams degummed silk cocoons in 250 grams of LiBr and 0.035 grams of Dithiothreitol in 300 mL of water and the silk solution was kept at 40° C. for 4 hours. The silk solution was transferred into cellulose tubing and dialyzed against 300 mL of distilled water containing 0.035 grams of dithiothreitol at room temperature for 24 hours to remove salts. After dialysis, to the silk solution, 3 grams of Polyvinyl Alcohol (PVA) was added and mixed. When the silk solution reaches a gel-like state, it was poured over a plate surface and made into a hydrogel film as bandage.

This bandage was then applied to the wound. Two six-week-old Balb/c mice, weighing around 150 grams, were placed under anesthesia, and a 5 mm round wound was created on the back. The hydrogel films were then placed over the wounds for 9 days, with images of the wound taken daily to track the healing process. It was found that the hydrogel film had increased wound healing compared to control medical gauze, and that fluorescence intensity of the bandage had decreased over time. The fluorescent silk fibroin bandage had healed about 80% of the entire wound, while the control medical gauze had only healed about 46%.

### CONCLUSION

The study indicates that silk produced by silkworms who were fed carbon quantum dots has promising applications within biomedical fields. Three types of nontoxic CQDs were synthesized through a hydrothermal method from Rhodamine B. These CQDs exhibited bright fluorescence and were successfully fed to silkworms, resulting in the production of fluorescent silk. Dissection of the silkworms revealed that the fluorescence was absorbed and traveled throughout their bodies, ultimately reaching the silk glands. Both the silk glands and almost all internal organs of the silkworm exhibited bright fluorescence, leading to the production of fluorescent cocoons. Additionally, structural studies of silk fibroin proved that silk produced by CQDs had shown a slightly thicker structure compared to control group silk: the CQD silk was about 2.23 nm thicker than control group. The nontoxic nature of fluorescent silk made from CQDs allowed for more biomedical applications as well. Mouse embryo-derived mesenchymal stem cells (MSCs) (C3H/10T1/2, Clone 8) were successfully grown within cell scaffolds, and it was shown that not only was fluorescent silk biocompatible, but also a great material for bioimaging as cells could be clearly identified. For the wound healing experiment, silk fibroin films were successfully fabricated by mixing polyvinyl alcohol and curcumin within the silk fibroin solution. The films were fabricated into bandages and placed over an about 5 mm wound on a Balb/c mouse. It was found that it had decreased the time it took for wounds to heal, with the bandage healing about 80% of the entire wound while the control had only healed about 46%. It was also found that the fluorescence within the bandage had lessened in intensity as time had passed, making it a great marker for the wound healing process. Overall, the study highlights the potential of fluorescent silk, particularly from CQDs, as a versatile material for a variety of biomedical applications, from tissue engineering to wound healing and bioimaging.

1. A fluorescent silk, wherein the fluorescent silk is produced by a process comprising feeding silkworms with a feed comprising a color carbon quantum dots (CQDs).

2. The fluorescent silk of claim 1, wherein the color CQDs is orange carbon quantum dots (CQDs), green CQDs or blue CQDs.

3. The fluorescent silk of claim 2, wherein the orange CQDs is prepared by hydrothermal treatment of a mixture of Rhodamine B and water at 240° C. for 13 hours.

4. The fluorescent silk of claim 2, wherein the green CQDs is prepared by hydrothermal treatment of a mixture of Rhodamine B, sodium hydroxide and water at 180° C. for 10 hours.

5. The fluorescent silk of claim 2, wherein the blue CQDs is prepared by hydrothermal treatment of a mixture of Rhodamine B, urea and water at 180° C. for 10 hours.

6. A biomedical product comprising the fluorescent silk of claim 1.

7. The biomedical product of claim 6, wherein the biomedical product is a fluorescent scaffold.

8. The biomedical product of claim 6, wherein the biomedical product is a bandage.

9. The biomedical product of claim 8, wherein the bandage is a hydrogel film comprising the fluorescent silk and a biocompatible polymer selected from the group consisting of polyvinyl alcohol, polyethylene oxide (PEO), polyethylene glycol (PEG), fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans and polyanhydrides and mixture thereof.

10. The biomedical product of claim 9, wherein the biocompatible polymer is polyvinyl alcohol.

11. The biomedical product of claim 9, wherein the fluorescent silk is at amount of 0.01% to 30% by weight of hydrogel film.

12. The biomedical product of claim 11, wherein the fluorescent silk is at amount of 0.1% to 10% by weight of hydrogel film.

13. The biomedical product of claim 9, wherein the biocompatible polymer is at amount of 0.01% to 30% by weight of hydrogel film.

14. The biomedical product of claim 13, wherein the biocompatible polymer is at amount of 0.1% to 10% by weight of hydrogel film.

15. The biomedical product of claim 9, wherein the fluorescent silk is at amount of 0.1% to 10% by weight of hydrogel film, and the biocompatible polymer is at amount of 0.1% to 10% by weight of hydrogel film.

16. A process of producing a fluorescent silk of claim 1 comprises feeding silkworms with a feed comprising a color carbon quantum dots (CQDs); and degumming obtained raw silk.

17. The process of claim 16, wherein the color CQDs is orange carbon quantum dots (CQDs), green CQDs or blue CQDs.

18. The process of claim 17, wherein the orange CQDs is prepared by hydrothermal treatment of a mixture of Rhodamine B and water at 240° C. for 13 hours.

19. The process of claim 17, wherein the green CQDs is prepared by hydrothermal treatment of a mixture of Rhodamine B, sodium hydroxide and water at 180° C. for 10 hours.

20. The process of claim 17, wherein the blue CQDs is prepared by hydrothermal treatment of a mixture of Rhodamine B, urea and water at 180° C. for 10 hours.
